(19) United States
(12) Patent Application Publication
(10) Pub. No.: US 2009/0012005 A1
(43) Pub. Date: Jan. 8, 2009

(54) Use of an angiotensin IV antagonist to treat insulin resistance or cardiovascular risk related to metabolic syndrome

(76) Inventors: Giampiero Bricca, Lyon (FR); Jacques Randon, Lyon (FR); Wassim Hodroj, Lyon (FR); Georges P. Vauquelin, Limal (BE); Dirk Tourwe, Itterbeek (BE)

Correspondence Address: CLARK & BRODY, 1090 VERMONT AVENUE, NW, SUITE 250, WASHINGTON, DC, 20005, US

(21) Appl. No.: 12/149,128

(22) Filed: Apr. 28, 2008

(30) Foreign Application Priority Data
Apr. 27, 2007 (FR) 07 54751

(51) Int. Cl. A61K 38/08; A61K 38/06; A61P 3/10; A61P 9/00

(52) U.S. Cl. 514/17; 514/18

(57) ABSTRACT

The present invention relates to the use of an angiotensin IV antagonist that decreases the effects on angiotensinogen expression of angiotensin IV in vascular smooth muscle cells for preparing a drug to treat or prevent type II diabetes, insulin resistance or cardiovascular risk related to metabolic syndrome, as well as to pharmaceutical compositions comprising one such antagonist.

### FIELD OF THE INVENTION

The present invention relates to the technical field of treating and preventing insulin resistance, in particular within the framework of treating type II diabetes, and cardiovascular risk related to metabolic syndrome. In particular, the invention relates to the use of an angiotensin IV antagonist, or a pharmaceutically acceptable salt of same, for preparing a drug to treat or prevent insulin resistance or cardiovascular risk related to metabolic syndrome, as well as pharmaceutical compositions comprising one such antagonist or pharmaceutically acceptable salt of same.

### BACKGROUND ART

Angiotensin IV is a natural product of higher organisms. It is the 3-8 peptide (Val-Tyr-Ile-His-Pro-Phe) of angiotensinogen. Angiotensin IV is produced by the successive action of various proteolytic enzymes on the only known angiotensinogen precursor. Starting with angiotensin II decapeptide, after the action of renin and conversion enzyme in the classic pathway, or other enzymes (tissue kallikrein, cathepsins D and G, etc., chymase) in alternate pathways, aminopeptidases A and N (as illustrated in FIG. 1) are the determining enzymes. The meaning of the abbreviations found in FIG. 1, according to Santos R A et al. 2000, are ACE, angiotensin conversion enzyme; Amp, aminopeptidase; Amp A, aminopeptidase A; Amp N, neutral aminopeptidase; D-Amp, dipeptidytaminopeptidase; Cbp, carboxypeptidase; EP N, neutral endopeptidase 24.11 (neprylsin); ECAZ, prolylcarboxypeptidase, ECA homolog; PEP prolylendopeptidase. The peptides with biological activity in FIG. 1 are marked by cross hatching in the boxes.

Aminopeptidases A and N are enzymes of the metalloprotease family and are present in a number of isoforms in various tissues. These isoforms may exhibit substrate characteristics as well as specific tissue distributions. At present, they are more often regarded as factors that either limit the local activity of hormones normally degraded by these enzymes or that, on the contrary, allow their production (¹⁻⁴). Angiotensin IV may be degraded by aminopeptidase N, most notably that produced by rat renal mesangial cells.

Plasma angiotensin IV levels measured in man, roughly 6 pg/ml to 8 pg/ml, may increase by a factor of five and may remain at elevated levels in response to treatment by an angiotensin II AT1 receptor antagonist(⁵).

Angiotensin IV receptors were initially characterized as angiotensin IV specific binding sites in the central nervous system and in various tissues and cells, in particular in the kidney, heart and arterial wall(⁶⁻¹²). The identification of insulin-regulated aminopeptidase (IRAP) as a likely angiotensin IV receptor marked an important step(¹³) IRAP is a membrane protein also known by other names such as oxytocinase, vp165, P-LAP, etc. It is a 165 kD membrane protein expressed in many cell types covering in large part the tissues in which binding studies have identified angiotensin IV receptors. IRAP belongs to the Ml aminopeptidase family. Angiotensin IV binds with IRAP at nanomolar concentrations, undoubtedly on the apoenzyme. At higher concentrations, angiotensin IV also inhibits IRAP enzymatic activity. IRAP, as its name indicates, is associated with the insulin response. Indeed, in the cell IRAP is highly colocalized with GLUT4 protein, which is responsible for the active transport of glucose in cells in response to insulin. Mice deficient in IRAP exhibit a decrease in GLUT4 transporter but maintain glucose homeostasis(¹⁴). Thus, today IRAP is often regarded as a protein required for transporting intracellular vesicles to the plasma membrane.

Knowing that angiotensin IV also acts as an inhibiter of IRAP enzymatic activity, two main interpretations of the biological effects of angiotensin IV have been proposed:

1. The effects of angiotensin IV are reflected in the inhibition of IRAP proteolytic activity and the effects observed are thus consistent with changes in the levels of peptides normally degraded by IRAP: angiotensin III, vasopressin, enkephalins, oxytocin, somatostatin, etc.

2. Angiotensin IV acts as a signal molecule that produces cellular effects via a membrane receptor and a transduction system. This receptor may be IRAP, although it does not belong to any known receptor family. But IRAP has the characteristic in its aminopeptidase family of having the intracellular portion as the most important, which is the portion likely to interact with, among others, the Akt substrate (AS160).

Nevertheless, older results using opossum kidney cell lines showed sensitivity of the bond between angiotensin IV and its receptor, which is characteristic of seven-transmembrane segment receptors coupled with G proteins(¹⁵) This result has not been confirmed in other cell types.

To date, several biological effects of angiotensin IV have been demonstrated.

At high concentrations, greater than μM, angiotensin IV may bind to angiotensin II AT1 receptors and produce effects similar to those of angiotensin II. These effects are thus blocked by angiotensin II AT1 receptor antagonists. The effects of angiotensin IV that might involve angiotensin II AT2 receptors have also been described, but, as is the case with AT1 receptors, these appear only at greater than μM angiotensin IV concentrations. The effects of angiotensin IV itself are thus defined either as effects identical to those obtained with angiotensin II, but insensitive to angiotensin II AT1 and AT2 receptor antagonists, or as different effects from those obtained by angiotensin II.

In the central nervous system, angiotensin IV administered by intracerebral route has demonstrated its ability to improve training and memorization performance in the rat(¹⁶⁻²⁰). More recent data, which need to be confirmed, suggest than angiotensin IV may play a protective role in cerebral vascular accidents via a vasodilator effect in the peripheral regions of the ischemic area(^{21;22}), which are likely to be prevented by co-administering an angiotensin II AT2 receptor antagonist such as PD 123319 or divalinal.

In cardiovascular system tissues and cells, the direct vascular effects of angiotensin IV have been studied in comparison with those of angiotensin II or III and with angiotensin II AT1 and AT2 receptor antagonists. In animals (rat, dog) and in man, angiotensin IV at concentrations equimolar with that of angiotensin II does not lead to changes in blood pressure or heart rate(²³). On particular isolated vascular beds, angiotensin IV at high concentrations may cause vasoconstrictor effects that are then dependent on the involvement of AT1 receptors. These vasoconstrictor effects could also arise by inhibition of endothelin degradation by IRAP. Vasodilator effects of angiotensin IV, independent of angiotensin II receptors and more difficult to demonstrate, have been described in mesenteric, cochlear, cerebral and renal vascular beds. Data concerning the involvement of endothelial cells in this effect are contradictory. In cultured smooth muscle cells, angiotensin IV, like angiotensin II but independent of angiotensin II AT1 receptors and by a mechanism blocked by divalinal (see below), stimulates NF-kB transcription factor and expression of genes dependent on it (²⁴). In the heart, angiotensin IV has specific binding sites on fibroblasts whose proliferation it may stimulate(^{25;26}). Angiotensin IV receptors are found in both vascular and pulmonary endothelial cells. In the former, angiotensin IV may, like angiotensin II, stimulate PAI-1 expression by a mechanism independent of angiotensin II AT1 or AT2 receptors. In the latter, it modifies phosphorylation of transcription factor 4E-BP1(^{11;27-30}).

Angiotensin IV receptors have also been described in the kidney(^{12;15;31-41}), in which angiotensin IV may have natriuretic effects. It also stimulates the proliferation of mesangial cells.

Identification of angiotensin IV binding sites was quickly followed by activity-structure correlation studies of angiotensin IV bond inhibition. The activity-structure correlations that have been established rely on inhibition of the angiotensin IV bond either with tissue or cell preparations of the receptor, or on cells that overexpress IRAP. Functionally, the initial tests were based on reproducing or blocking the effects of angiotensin IV in the central nervous system and in other situations in which the specific effects of angiotensin IV have been observed. Since the identification of IRAP as a likely angiotensin IV receptor, activity-structure correlation studies have also been focused on the inhibition of aminopeptidase activity (^{20;42-52}). The properties of several peptides derived from the structure of angiotensin IV have been studied subsequently.

From the point of view of the effects of angiotensin IV that contrast with the deleterious effects of angiotensin II, especially for its effects on memory and learning processes, agonist ligands have been sought in particular(^{19;53-55}).

Divalinal [N-Val-(CH₂—NH)-Tyr-Val(CH₂—NH)-His-Pro-Phe] has also appeared to be an angiotensin IV antagonist that inhibits some of its effects. The effects of divalinal on those produced by angiotensin IV are, however, highly diverse since potentiation as well as inhibition or an absence of interactions have also been found.

### SUMMARY OF THE INVENTION

Within the framework of the invention, the inventors are more particularly interested in the effects of angiotensin IV in vascular smooth muscle cells in comparison with those of angiotensin II on the expression of a certain number of genes coding for proteins implicated in a local renin-angiotensin system as well as in the insulin response. They have shown that angiotensin IV exerts effects opposite those of angiotensin II on the expression of a certain number of genes, including angiotensinogen and AT1 receptors (AT1R), at nanomolar concentrations by mechanisms independent of those involved with angiotensin II.

By using the insulin-induced 3H-deoxyglucose uptake technique, the inventors have also demonstrated that, in the same cells, angiotensin IV acts as a powerful inhibiter of insulin-induced glucose uptake (ED₅₀ of roughly 11 nM). This effect by angiotensin IV is not blocked by angiotensin II AT1 receptor antagonists or by angiotensin II AT2 receptor antagonists.

Thus, these results demonstrate the existence of a physiological process by which endogenous angiotensin IV up-regulates the expression of angiotensinogen and AT1 receptors, but down-regulates cell sensitivity to insulin and to IRAP expression. Hyperstimulation of this system is thus likely to participate in the genesis of insulin resistance and thus that of type II diabetes. Activation by angiotensin IV of angiotensinogen expression and thus of angiotensin II production as well as activation of angiotensin II AT1 receptor expression and thus smooth muscle cell sensitivity contribute to this effect because angiotensin II in this cell type also inhibits the insulin response in terms of glucose uptake.

Finally, the inventors have shown that angiotensin IV antagonists, capable of decreasing the biological activity of angiotensin IV either by modifying its ED₅₀ or by decreasing its maximum effect, may be used to treat or prevent insulin resistance, in particular within the framework of treating type II diabetes or cardiovascular risk related to metabolic syndrome.

Thus, the invention relates to the use of an angiotensin IV antagonist that decreases the effects on angiotensinogen expression of angiotensin IV in vascular smooth muscle cells for preparing a drug to treat or prevent type II diabetes, insulin resistance or cardiovascular risk related to metabolic syndrome.

### BRIEF DESCRIPTION OF THE DRAWINGS

FIG. 1 is a flow chart showing the production of angiotensin IV from angiotensinogen.

FIG. 2A is a graph representing dose response correlation of angiotensin II and angiotensin IV against angiotensin.

FIG. 2B is a graph representing dose response correlation of angiotensin II and angiotensin IV against AT1 receptors.

FIG. 2C is a graph representing the effect of an AT1 antagonist on angiotensin II and angiotensin IV.

FIG. 3 is a graph representing the inhibition of the angiotensinogen response to angiotensin IV by divalinal and compound AL-11.

FIG. 4 is a graph demonstrating inhibition of insulin-induced deoxyglucose uptake in human vascular smooth muscle cells by angiotensin IV.

FIG. 5 is a graph demonstrating the reversion obtained with two AT IV antagonists of the inhibition of insulin-induced deoxyglucose uptake in human vascular smooth muscle cells by angiotensin IV.

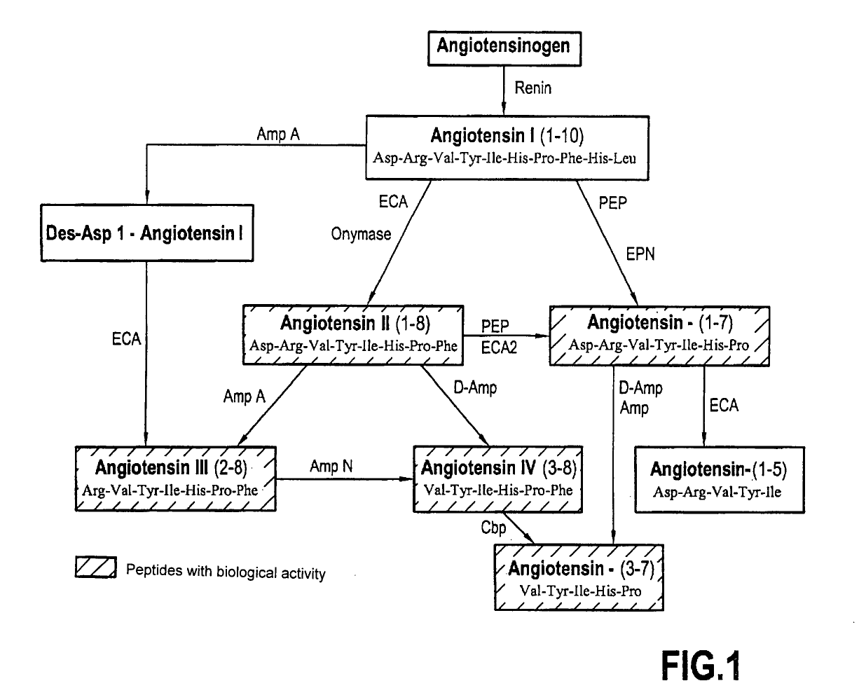

### DESCRIPTION OF THE PREFERRED EMBODIMENTS

Within the framework of the invention, “angiotensin IV antagonist” means a compound capable of binding to an angiotensin IV receptor and preventing the binding of angiotensin IV to this receptor, thus reducing the biological activity of angiotensin IV via this receptor. This reduction in biological activity may, for example, appear as an increase in its ED₅₀, i.e., by reducing its maximum effect. In particular, angiotensin IV antagonist decreases the biological effects of angiotensin IV produced by a mechanism independent of angiotensin II AT1 and AT2 receptors. Such activity can, for example, be demonstrated by studying gene expression in the renin-angiotensin system in primary human smooth muscle cell cultures, for example according to the test described below. The compounds used within the framework of the invention decrease the effects of angiotensin IV angiotensinogen expression in vascular smooth muscle cells. Advantageously, the antagonist increases the angiotensin IV ED₅₀ by at least a factor of 10 or a reduction of at least 50% of its maximum effect obtained by varying the concentration of angiotensin IV in said test.

Examples of such antagonists include divalinal of formula (1):

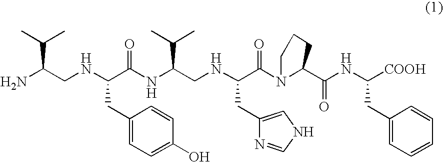

and compound AL-11: H-β²hVal-Tyr-Ile-His-Pro-β³hPhe-OH, of formula (2), prepared according to classic peptide synthesis methods:

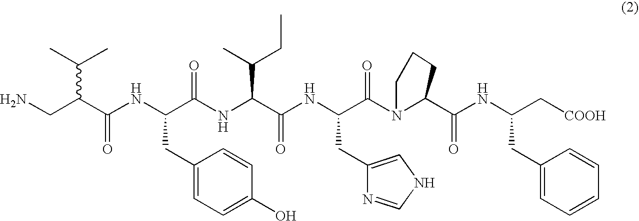

Divalinal, for example, may be synthesized according to the method described by M. F. Sardinia et al., in Peptides 1994, 15(8), 1399-1406.

AL-11 may be synthesized by solid-phase peptide synthesis using amino acids protected at the N-terminal by N-9-fluorenylmethoxycarbonyl (Fmoc) and 2-chlorotrityl chloride resin (1.5 mmol/g). The protective groups for the side chains are Tyr(t-Bu) and H is(Trt). The Fmoc protective group is eliminated with 20% piperidine in DMF (2×5 min). The amino acids are coupled in 1:1 DMF/CH₂Cl₂ (v/v) in the presence of O-(Benzotriazol-1-yl)-N,N,N′,N′-tetramethyluronium tetrafluoroborate (TBTU, 3 eq.) and diisopropylethylamine (DIPEA, 6 eq.). The peptide is separated from the resin by treatment with a triethylsilane/H₂O/trifluoroacetic acid mixture (95:2.5:2.5) for two hours and then purified by RP-HPLC on a Unipoint software controlled Gilson apparatus and a SUPELCO Discovery® BIO Wide Pore C18 preparative column (25 cm×21.2 mm, 10 μm). The mobile phase is comprised of a water/acetonitrile mixture containing 0.1% TFA. The product is eluted using a 3% to 97% acetonitrile gradient over 20 minutes at 20 ml/min. The structure of the compound was confirmed by mass spectrometry.

The antagonists of the invention may be in the form of a pharmaceutically acceptable salt.

Pharmaceutically acceptable salts include physiologically acceptable acid or base salts and organic or mineral salts. Suitable acids include hydrochlorides, hydrobromides, sulfates, hydrogen sulfates, dihydrogen phosphates, maleates, fumarates, 2-naphthalenesulfonates and paratoluenesulfonates. Base salts include sodium salts or potassium salts with a physiologically acceptable amino, etc. Such salts are prepared according to techniques well known to those persons skilled in the art.

The pharmaceutical compositions of the invention contain an effective dose of an angiotensin IV antagonist in the form of a pharmaceutically acceptable salt, solvate or hydrate in combination with at least one suitable excipient. The excipient or excipients and the vehicle for the composition are selected according to the dosage form and the desired administration route. Oral or injectable administration is preferred.

In the pharmaceutical compositions of the invention for oral, sublingual, subcutaneous, intramuscular, intravenous, topical, intratracheal, intranasal, transdermal, rectal or intraocular administration, the angiotensin IV antagonist, in the form of a salt, solvate or hydrate, may be administered in dosage unit forms of administration, in mixtures with classic pharmaceutical carriers, in animals and in humans to prevent or treat insulin resistance or cardiovascular risk related to metabolic syndrome. Suitable dosage unit forms of administration include, in particular, forms by oral route such as tablets, gelatin capsules, powders, granules and oral solutions or suspensions; sublingual, buccal, intratracheal and intranasal administration forms; subcutaneous, intramuscular and intravenous administration forms; and rectal administration forms.

In order to obtain the prophylactic or therapeutic effect desired, each unit dose may contain from 0.1 mg to 500 mg of angiotensin IV antagonist in combination with a pharmaceutical carrier. This unit dose may be administered 1 to 5 times per day in such a way as to administer a daily dosage that achieves the desired effect.

To prepare a solid composition in tablet form, the principal active ingredient is mixed with a pharmaceutical vehicle such as gelatin, starch, lactose, magnesium stearate, talc, gum arabic or similar. The tablets may be coated with sucrose, cellulose derivative or another suitable substance or they may be treated in such a way as to exhibit extended or delayed activity and to continuously release a predetermined quantity of active ingredient.

Gelatin capsules may be prepared by mixing the active ingredient with a diluent and then pouring the mixture obtained into soft or hard gelatin capsules.

The pharmaceutical compositions containing an angiotensin IV antagonist may also be provided in liquid form, for example, solutions, emulsions, suspensions or syrups. Suitable liquid carriers may be, for example, water, organic solvents such as glycerol or glycols, as well as mixtures thereof, in varied proportions in water.

A preparation in syrup or elixir form for dropwise administration may contain the active ingredient together with a sweetener, preferably calorie-free, methylparaben and propylparaben as an antiseptic, as well as a flavoring agent and a suitable colorant. Powders or granules to be dissolved in water may contain the active ingredient in a mixture with dispersants or wetting agents, or suspension agents such as polyvinylpyrrolidone, as well as with sweeteners or flavoring agents.

Suppositories are used for rectal administration and are prepared with binders that melt at rectal temperature, for example cocoa butter or polyethylene glycols. For parenteral administration, options include aqueous suspensions, isotonic saline solutions or sterile and injectable solutions containing pharmacologically compatible dispersants and/or wetting agents, for example propylene glycol or butylene glycol. The active ingredient may be also formulated as microcapsules, optionally with one or more carriers or additives, or with matrices such as a polymer or cyclodextrin (extended release patches).

In addition to the angiotensin IV antagonist, the compositions of the invention may contain, for example, active ingredients that may be useful to treat or prevent insulin resistance or cardiovascular risk related to metabolic syndrome.

Thus, the present invention also relates to pharmaceutical compositions containing several active ingredients in combination, of which one is an angiotensin IV antagonist or a pharmaceutically acceptable salt thereof.

The antagonist activity of the compounds tested was demonstrated according to biological tests in which angiotensin IV produced specific and selective effects. The specificity of the effects of angiotensin IV on gene expression in the renin-angiotensin system in cells in culture (primary human smooth muscle cell cultures, rat cell lines) meets these criteria. The inventors have shown that for a variety of genes, including those for angiotensinogen, AT1 receptor, IRAP and aminopeptidase N, angiotensin IV at nanomolar concentrations exerts effects opposite those of angiotensin II. These effects of angiotensin IV are neither modified by angiotensin II AT1 receptor antagonists, nor by angiotensin II AT2 receptor antagonists, nor by combinations thereof.

Such a test is thus specific because only angiotensin IV produces this stimulatory effect, whereas angiotensin II inhibits the expression of these genes. This effect may be measured by analyzing messenger RNA as well as the corresponding proteins.

Inhibition of these effects of angiotensin IV by candidate compounds is the foundation of the model. These cellular models encompass a screening approach as well as quantitative approaches. Angiotensin IV is used at a concentration of 100 nM, a concentration at which its binding to angiotensin II receptors is minimal.

The therapeutic activity of the compounds tested was demonstrated using a test based on restoration of insulin sensitivity in cells treated with angiotensin IV. Angiotensin IV inhibits insulin-induced glucose uptake in vascular smooth muscle cells by a mechanism independent of angiotensin II AT1 and AT2 receptors. The inventors demonstrated that this mechanism proceeds by phosphorylation dependent on the MAP-kinase pathway of IRS1 serines 312 and 616, leading to inhibition of activation of the p85 AKT pathway, translocation of GLUT4 and glucose uptake. Inhibition of the effects of angiotensin IV by the compounds tested demonstrates their therapeutic potential.

The inventors used primary cultures and vascular muscle smooth cell lines of various species (man, rat, mouse); all cell lines in which angiotensin IV exerts stimulatory effects on the expression of angiotensinogen and angiotensin II AT1 receptors may be used. The confluent smooth muscle cells are placed in a serum-free culture medium (human vascular smooth muscle medium 2 from Promocell, or any other equivalent medium), in order to block their multiplication and to synchronize them. After 24 hours of habituation in this culture medium, the medium is replaced by fresh medium to which the various test substances are added according to the order adapted to the experimental protocol. Measurements are taken after 24 hours of treatment in the case of analyses of angiotensinogen and AT1 receptor expression. The antagonists are applied to the cells 30 minutes before angiotensin IV. The cells are exposed to 100 μU/ml of insulin to measure glucose uptake.

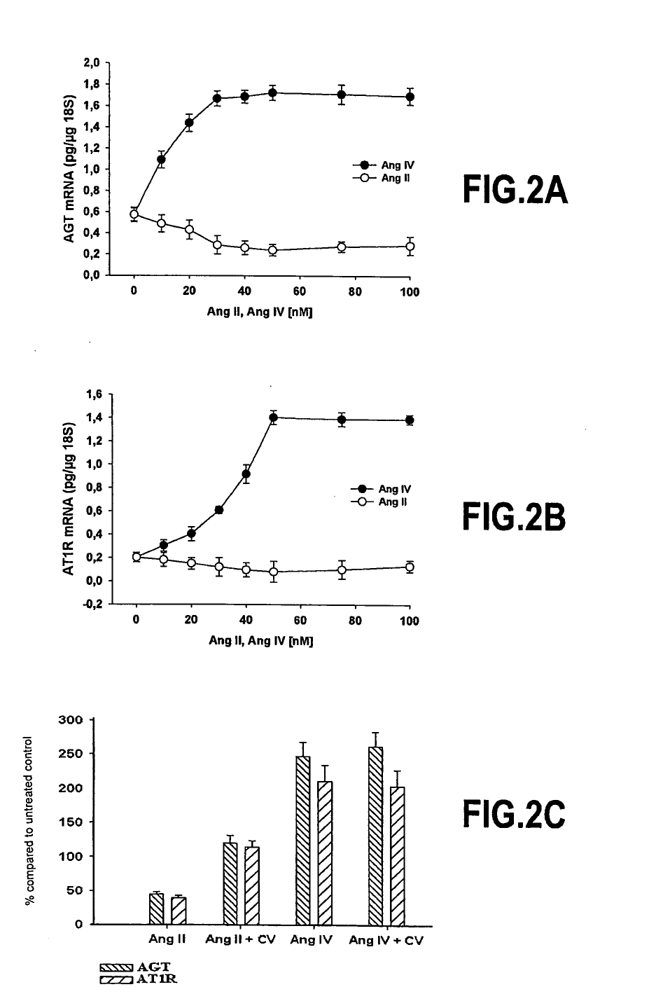

Thus, the inventors were able to show that angiotensin IV exerts powerful effects that are opposite those of angiotensin II and independent of AT1 receptors stimulating the expression of angiotensinogen and AT1 receptors, whereas it reduces IRAP expression. FIG. 2 presents the dose-response correlation of angiotensin II (Ang II) and angiotensin IV on angiotensinogen (AGT) mRNA levels (plot A) and on AT1 receptors (AT1R) (plot B). The results are the mean of pairs of measurements taken in human vascular smooth muscle cells from six patients. Plot C shows that the effects of 100 nM angiotensin II are blocked by an AT1 antagonist and that, in contrast, an AT1 antagonist does not block the effects of 100 nM angiotensin IV.

The ED₅₀ (dose that yields 50% of the maximum effect) of angiotensin IV analyzed in the models for its effects on various genes is consistently on the order of 10 nM to 20 nM (plot A and B of FIG. 2). These effects of angiotensin IV are not blocked by an angiotensin II AT1 receptor antagonist, contrary to those induced by angiotensin II itself (FIG. 2 plot C).

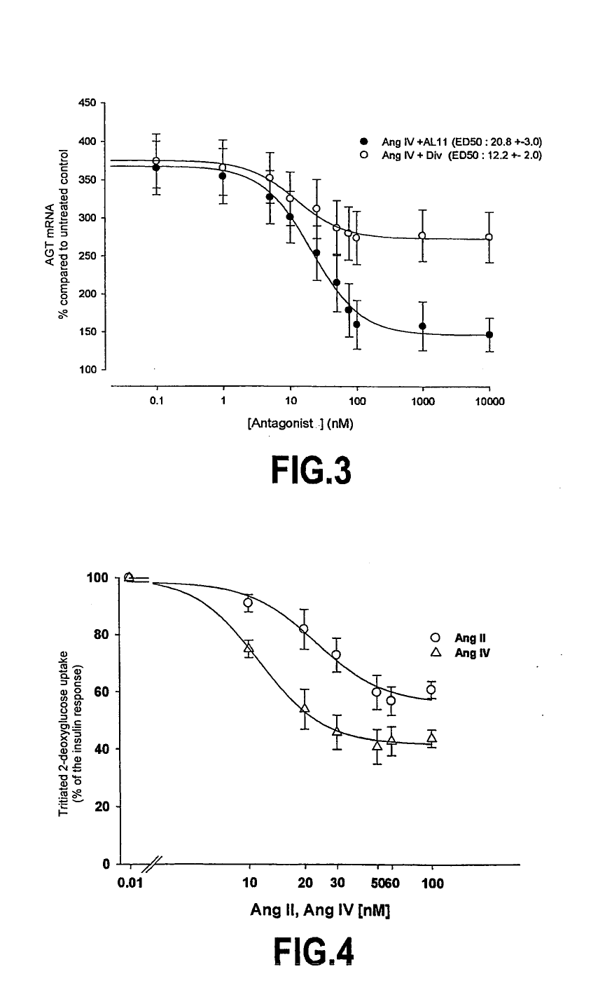

FIG. 3 demonstrates inhibition of the angiotensinogen response to 100 nM angiotensin IV by divalinal and compound AL-11 at increasing concentrations, administered 30 minutes before angiotensin IV. The key for Ang II and Ang IV for FIG. 4 is Angiotensin II (4-parameter logistic regression: amplitude of maximum effect=42.2%±6.4%, slope coefficient: nH: 2.39±0.90, inflection point: ED50=23.1±3.8 nM; lower plateau: 56.3±4.5) and Ang IV (4-parameter logistic regression: amplitude of maximum effect=58.0%±2.3%, slope coefficient: nH:2.51±0.36, ED50=11.3±0.62 nM; lower plateau: 41.9±1.4).

FIG. 4 demonstrates inhibition, by increasing concentrations of angiotensin IV, of insulin-induced deoxyglucose uptake in human vascular smooth muscle cells. The results are the mean of pairs of measurements taken in cells from six patients. Similar results were obtained with a rat vascular smooth muscle cell line.

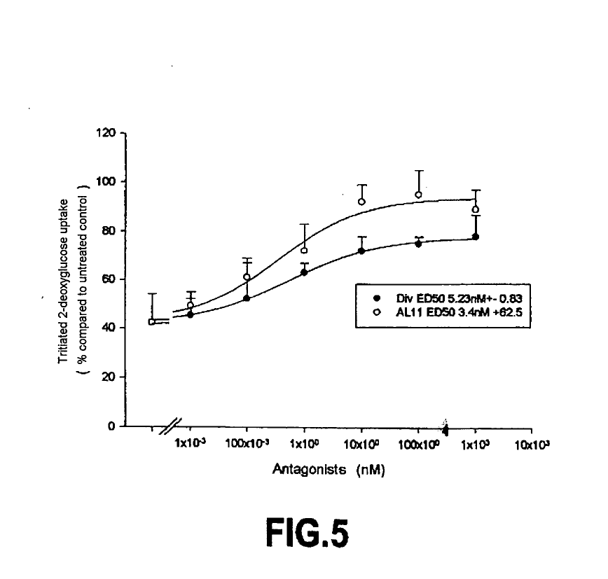

The inventors also demonstrated that the inhibitory effects of angiotensin IV on insulin-induced glucose uptake may be prevented by the compounds that exhibited antagonist activity in the preceding test. FIG. 5 demonstrates the reversion obtained with two AT IV antagonists (divalinal and compound AL-11) of the inhibition by angiotensin IV of insulin-induced glucose uptake in human vascular smooth muscle cells. The results are the mean of pairs of measurements taken in cells from five patients. These results also show that under the experimental conditions used (pretreatment and not simultaneous application) divalinal behaves as a weaker depressor of the maximum response of angiotensin IV and that compound AL-11 is much more powerful in preventing the response in AGT genes and AT1 receptors, as highlighted in FIG. 3, as well as in the insulin response. LVV-hemorphin-7 displayed no activity in these various tests.

### REFERENCES

1. Banegas I, Prieto I, Vives F et al. Brain aminopeptidases and hypertension. J Renin Angiotensin Aldosterone Syst 2006; 7:129-134.
2. Carl-McGrath S, Lendeckel U, Ebert M et al. Ectopeptidases in tumour biology: a review. Histol Histopathol. 2006; 21:1339-1353.
3. Walling L L. Recycling or regulation? The role of amino-terminal modifying enzymes. Curr Opin Plant Biol. 2006; 9:227-233.
4. Tsujimoto M, Hattori A. The oxytocinase subfamily of Ml aminopeptidases. Biochim BiophysActa. 2005; 1751:9-18.

5. Shibasaki Y, Mori Y, Tsutumi Y et al. Differential kinetics of circulating angiotensin IV and II after treatment with angiotensin II type 1 receptor antagonist and their plasma levels in patients with chronic renal failure. Clin Nephrol. 1999; 51:83-91.
6. Hanesworth J M, Sardinia M F, Krebs L T et al. Elucidation of a specific binding site for angiotensin 11(3-8), angiotensin IV, in mammalian heart membranes. J Pharmacol Exp Then 1993; 266:1036-1042.
7. Hail K L, Hanesworth J M, Ball A E et al. Identification and characterization of a novel angiotensin binding site in cultured vascular smooth muscle cells that is specific for the hexapeptide (3-8) fragment of angiotensin II, angiotensin IV. ReguiPept 1993; 44:225-232.
8. Jarvis M F, Gessner G W, Ly C Q. The angiotensin hexapeptide 3-8 fragment potently inhibits [125I]angiotensin II binding to non-AT1 or -AT2 recognition sites in bovine adrenal cortex. Eur J. Pharmacol. 1992; 219:319-322.
9. Swanson G N, Hanesworth J M, Sardinia M F et al. Discovery of a distinct binding site for angiotensin II (3-8), a putative angiotensin IV receptor. Regul Pept 1992; 40:409-419.
10. Czekalski S, Chansel D, Vandermeersch S et al. Evidence for angiotensin IV receptors in human collecting duct cells. Kidney Int. 1996; 50:1125-1131.
11. Riva L, Galzin A M. Pharmacological characterization of a specific binding site for angiotensin IV in cultured porcine aortic endothelial cells. Eur J. Pharmacol. 1996; 305:193-199.
12. Coleman J K, Krebs L T, Hamilton T A et al. Autoradiographic identification of kidney angiotensin IV binding sites and angiotensin IV-induced renal cortical blood flow changes in rats. Peptides. 1998; 19:269-277.
13. Albiston A L, McDowall S G, Matsacos D et al. Evidence that the angiotensin IV (AT(4)) receptor is the enzyme insulin-regulated aminopeptidase. J Biol. Chem. 2001; 276:48623-48626.
14. Keller S R, Davis A C, Clairmont K B. Mice deficient in the insulin-regulated membrane aminopeptidase show substantial decreases in glucose transporter GLUT4 levels but maintain normal glucose homeostasis. J Biol. Chem. 2002; 277:17677-17686.
15. Dulin I S I, Madhun Z T, Chang C H et al. Angiotensin IV receptors and signaling in opossum kidney cells. Am J. Physiol. 1995; 269:F644-F652.
16. Braszko J J, Walesiuk A, Wielgat P. Cognitive effects attributed to angiotensin II may result from its conversion to angiotensin IV. J Renin Angiotensin Aldosterone Syst 2006; 7:168-174.
17. Chai S Y, Fernando R, Peck G et al. The angiotensin IV/AT4 receptor. Cell Mol. LifeSci. 2004; 61:2728-2737.
18. Albiston A L, Fernando R, Ye S et al. Alzheimer's, angiotensin IV and an aminopeptidase. Biol Pharm Bull. 2004; 27:765-767.
19. Wright J W, Harding J W. The brain angiotensin system and extracellular matrix molecules in neural plasticity, learning, and memory. Prog Neurobiol. 2004; 72:263-293.
20. Lee J, Albiston A L, Allen A M et al. Effect of I.C.V. injection of AT4 receptor ligands, NLEl-angiotensin IV and LW-hemorphin 7, on spatial learning in rats. Neuroscience. 2004; 124:341-349.
21. Faure S, Chapot R, Tallet D et al. Cerebroprotective effect of angiotensin IV in experimental ischemic stroke in the rat mediated by AT(4) receptors. J Physiol Pharmacol. 2006; 57:329-342.
22. Faure S, Javellaud J, Achard J M et al. Vasoconstrictive effect of angiotensin IV in isolated rat basilar artery independent of AT1 and AT2 receptors. J Vase Res. 2006; 43:19-26.
23. Abrahamsen C T, Pullen M A, Schnackenberg C G et al. Effects of angiotensins II and IV on blood pressure, renal function, and PAI-1 expression in the heart and kidney of the rat. Pharmacology. 2002; 66:26-30.
24. Esteban V, Ruperez M, Sanchez-Lopez E et al. Angiotensin IV activates the nuclear transcription factor-kappaB and related proinflammatory genes in vascular smooth muscle cells. Circ Res. 2005; 96:965-973.
25. Wang L, Eberhard M, Erne P. Stimulation of DNA and RNA synthesis in cultured rabbit cardiac fibroblasts by angiotensin IV. Clin Sci (Lond). 1995; 88:557-562.
26. Wang L, Eberhard M, Kohler E et al. A specific binding site for angiotensin 11(3-8), angiotensin IV, in rabbit cardiac fibroblasts. J Recept Signal Transduct Res. 1995; 15:517-527.
27. Lu J, Zhang J, Block E R et al. Angiotensin IV enhances phosphorylation of 4EBP1 by multiple signaling events in lung endothelial cells. Mol Cell Biochem. 2005; 275:181-188.
28. Kerins D M, Hao Q, Vaughan D E. Angiotensin induction of PAI-1 expression in endothelial cells is mediated by the hexapeptide angiotensin IV. J Clin Invest 1995; 96:2515-2520.
29. Bernier S G, Servant G, Boudreau M et al. Characterization of a binding site for angiotensin IV on bovine aortic endothelial cells. Eur J. Pharmacol. 1995; 291:191-200.
30. Hall K L, Venkateswaran S, Hanesworth J M et al. Characterization of a functional angiotensin IV receptor on coronary microvascular endothelial cells. RegulPept. 1995; 58:107-115.
31. Handa R K. Biphasic actions of angiotensin IV on renal blood flow in the rat. RegulPept 2006; 136:23-29.
32. Li X C, Campbell D J, Ohishi M et al. ATI receptor-activated signaling mediates angiotensin IV-induced renal cortical vasoconstriction in rats. Am J Physiol Renal Physiol. 2006; 290:F1024-F1033.
33. Hamilton T A, Handa R K, Harding J W et al. A role for the angiotensin IV/AT4 system in mediating natriuresis in the rat. Peptides. 2001; 22:935-944.
34. Chansel D, Vandermeersch S, Oko A et al. Effects of angiotensin IV and angiotensin̂ 1-7) on basal and angiotensin II-stimulated cytosolic Ca2+ in mesangial cells. Eur J Pharmacol. 2001; 414:165-175.
35. Handa R K. Characterization and signaling of the AT(4) receptor in human proximal tubule epithelial (HK-2) cells. J Am Soc Nephrol. 2001; 12:440-449.
36. Handa R K, Harding J W, Simasko S M. Characterization and function of the bovine kidney epithelial angiotensin receptor subtype 4 using angiotensin IV and divalinal angiotensin IV as receptor ligands. J Pharmacol Exp Then 1999; 291:1242-1249.
37. Gesualdo L, Ranieri E, Monno R et al. Angiotensin IV stimulates plasminogen activator inhibitor-1 expression in proximal tubular epithelial cells. Kidney Int. 1999; 56:461-470.
38. Grove K L, Deschepper C F. High salt intake differentially regulates kidney angiotensin IV AT4 receptors in Wistar-Kyoto and spontaneously hypertensive rats. LifeSci. 1999; 64:1811-1818.
39. Kramar E A, Krishnan R, Harding J W et al. Role of nitric oxide in angiotensin IV-induced increases in cerebral blood flow. Regul Pept 1998; 74:185-192.
40. Handa R K, Krebs L T, Harding J W et al. Angiotensin IV AT4-receptor system in the rat kidney. Am J Physiol. 1998; 274: F290-F299.
41. Ardaillou R, Chansel D. [Angiotensin IV, a new component of the renin-angiotensin system, which acts on kidney cells]. Bull Acad Natl Med. 1996; 180:475-486.
42. Axen A, Lindeberg G, Demaegdt H et al. Cyclic insulin-regulated aminopeptidase (IRAP)/AT4 receptor ligands. J PeptSci. 2006; 12:705-713.
43. Lew R A, Mustafa T. Ye S et al. Angiotensin AT4 ligands are potent, competitive inhibitors of insulin regulated aminopeptidase (IRAP). J Neurochem. 2003; 86:344-350.
44. Lee J, Mustafa T, McDowall S G et al. Structure-activity study of LW-hemorphin-7: angiotensin AT4 receptor ligand and inhibitor of insulin-regulated aminopeptidase. J Pharmacol Exp Then 2003; 305:205-211.
45. Lee J, Chai S Y, Mendelsohn F A et al. Potentiation of cholinergic transmission in the rat hippocampus by angiotensin IV and LW-hemorphin-7. Neuropharmacology. 2001; 40:618-623.
46. Krishnan R, Hanesworth J M, Wright J W et al. Structure-binding studies of the adrenal AT4 receptor: analysis of position two- and three-modified angiotensin IV analogs. Peptides. 1999; 20:915-920.
47. Moeller I, Albiston A L, Lew R A et al. A globin fragment, LW-hemorphin-7, induces [3H]thymidine incorporation in a neuronal cell line via the AT4 receptor. J Neurochem. 1999; 73:301-308.
48. Garreau I, Chansel D, Vandermeersch S et al. Hemorphins inhibit angiotensin IV binding and interact with aminopeptidase N. Peptides. 1998; 19:1339-1348.
49. Moeller I, Chai S Y, Smith I et al. Haemorphin peptides may be endogenous ligands for brain angiotensin AT4 receptors. Clin Exp Pharmacol Physiol Suppl. 1998; 25:S68-S71.
50. Moeller I, Lew R A, Mendelsohn F A et al. The globin fragment LW-hemorphin-7 is an endogenous ligand for the AT4 receptor in the brain. J Neurochem. 1997; 68:2530-2537.
51. Sardinia M F, Hanesworth J M, Krishnan F et al. AT4 receptor structure-binding relationship: N-terminal-modified angiotensin IV analogues. Peptides. 1994; 15:1399-1406.
52. Sardinia M F, Hanesworth J M, Krebs L T et al. AT4 receptor binding characteristics: D-amino acid- and glycine-substituted peptides. Peptides. 1993; 14:949-954.
53. Goto Y, Hattori A, Ishii Y et al. Enzymatic properties of human aminopeptidase A. Regulation of its enzymatic activity by calcium and angiotensin IV. J BiolChem. 2006; 281:23503-23513.
54. Gard P R. Angiotensin as a target for the treatment of Alzheimer's disease, anxiety and depression. Expert Opin Trier Targets. 2004; 8:7-14.
55. Poljak A, McLean C A, Sachdev P et al. Quantification of hemorphins in Alzheimer's disease brains. J Neurosci Res. 2004; 75:704-714.

1. Use of an angiotensin IV antagonist that decreases the effects on angiotensinogen expression of angiotensin IV in vascular smooth muscle cells for preparing a drug to treat or prevent type II diabetes, insulin resistance or cardiovascular risk related to metabolic syndrome.

2. Use according to claim 1 characterized in that the angiotensin IV antagonist decreases the biological effects of angiotensin IV produced by a mechanism independent of angiotensin II AT1 and AT2 receptors.

3. Use according to claim 1 characterized in that the antagonist increases the ED₅₀ of the angiotensin or decreases its maximum effect on the expression of angiotensinogen and/or AT1 receptors in vascular smooth muscle cells.

4. Use according to claim 3 characterized in that the antagonist increases the angiotensin IV ED₅₀ by at least a factor of 10 or reduces by at least 50% its maximum effect on the expression of angiotensinogen and/or AT1 receptors.

5. Use according to claim 1 characterized in that the antagonist is a peptide compound.

6. Use according to claim 1 characterized in that the antagonist is divalinal or compound AL-11 of formula (2):

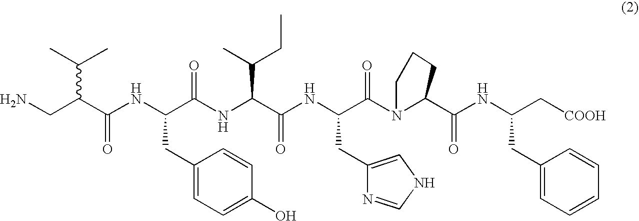

7. Use according to claim 2 characterized in that the antagonist increases the ED₅₀ of the angiotensin or decreases its maximum effect on the expression of angiotensinogen and/or AT1 receptors in vascular smooth muscle cells.

8. Use according to claim 2 characterized in that the antagonist is a peptide compound.

9. Use according to claim 3 characterized in that the antagonist is a peptide compound.

10. Use according to claim 4 characterized in that the antagonist is a peptide compound.

11. Use according to claim 2 characterized in that the antagonist is divalinal or compound AL-11 of formula (2):

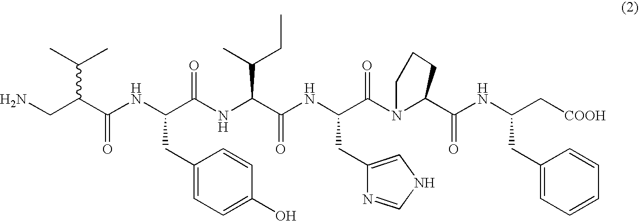

12. Use according to claim 3 characterized in that the antagonist is divalinal or compound AL-11 of formula (2):

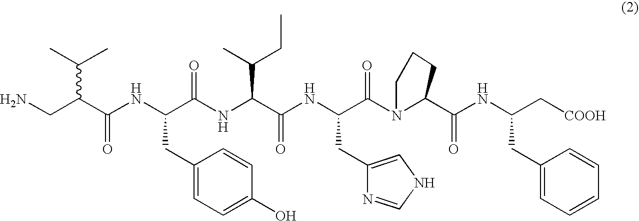

13. Use according to claim 4 characterized in that the antagonist is divalinal or compound AL-11 of formula (2):

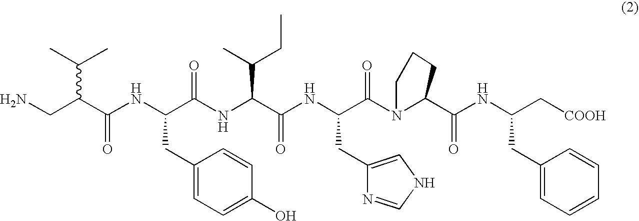

14. Use according to claim 5 characterized in that the antagonist is divalinal or compound AL-11 of formula (2): [chemical structure]
